(19) United States
(12) Patent Application Publication
(10) Pub. No.: US 2018/0344135 A1
(43) Pub. Date: Dec. 6, 2018

(54) SCANNING ENDOSCOPE SYSTEM

(71) Applicant: OLYMPUS CORPORATION, Tokyo (JP)

(72) Inventors: Soichiro KOSHIKA, Tokyo (JP); Kazuma KANEKO, Tokyo (JP)

(73) Assignee: OLYMPUS CORPORATION, Tokyo (JP)

(21) Appl. No.: 16/057,878

(22) Filed: Aug. 8, 2018

Related U.S. Application Data
(63) Continuation of application No. PCT/JP2016/083908, filed on Nov. 16, 2016.

(30) Foreign Application Priority Data
Feb. 12, 2016 (JP) 2016-024843

(51) Int. Cl. A61B 1/00; H04N 5/225; H04N 17/00; A61B 1/07

(52) U.S. Cl. CPC A61B 1/00172; H04N 5/2256; H04N 2005/2255; A61B 1/07; A61B 1/00006; H04N 17/002

(57) ABSTRACT

A scanning endoscope system includes an optical fiber for guiding illumination light, an actuator configured to displace an irradiation position of the illumination light emitted through the optical fiber, and a controller. The controller generates a driving signal having periodicity for driving the actuator and supplies the driving signal via a predetermined signal line connected to the actuator, determines presence or absence of occurrence of a trouble in the predetermined signal line based either on a current value of an electric current flowing in the predetermined signal line at predetermined timing in a period for one cycle of the driving signal or a voltage value of a voltage applied to the actuator at the predetermined timing, and obtains a determination result that a trouble has occurred in the predetermined signal line when detecting that the current value continuously deviates predetermined times from a predetermined threshold range.

### CROSS REFERENCE TO RELATED APPLICATION

This application is a continuation application of PCT/JP2016/083908 filed on Nov. 16, 2016 and claims benefit of Japanese Application No. 2016-024843 filed in Japan on Feb. 12, 2016, the entire contents of which are incorporated herein by this reference.

### BACKGROUND OF INVENTION

### 1. Field of the Invention

The present invention relates to a scanning endoscope system and, more particularly, to a scanning endoscope system that scans an object with light.

### 2. Description of the Related Art

In a medical field, for example, an electronic endoscope disclosed in Japanese Patent Application Laid-Open Publication No. 2-28967 has been used. More specifically, Japanese Patent Application Laid-Open Publication No. 2-28967 discloses an electronic endoscope configured to pick up, with a solid-state image pickup device such as a CCD, an image of an object illuminated with light supplied from a light source apparatus. Japanese Patent Application Laid-Open Publication No. 2-28967 discloses a configuration for detecting trouble as disconnection of a wire in a cable for connecting a solid-state image pickup device provided in a distal end camera section of an endoscope and a camera control unit configured to convert a signal from the solid-state image pickup device into a video signal and output the video signal to a display apparatus.

On the other hand, in the medical field, as an endoscope including a configuration different from the electronic endoscope explained above, in recent years, for example, an endoscope of a scanning type configured to scan an object in a body cavity of a subject with laser light to acquire an image has been proposed. More specifically, for example, the endoscope of the scanning type swings, according to operation of an actuator attached to an optical fiber for guiding laser light emitted from a light source, an end portion of the optical fiber to thereby displace an irradiation position of the laser light emitted through the end portion of the optical fiber and scan the object.

### SUMMARY OF THE INVENTION

A scanning endoscope system according to an aspect of the present invention includes: an optical fiber for guiding illumination light supplied from a light source section; an actuator configured to displace an irradiation position of the illumination light emitted from the optical fiber; and a controller configured to: generate a driving signal including periodicity as a signal for driving the actuator; supply the driving signal to the actuator via a predetermined signal line connected to the actuator; detect presence or absence of occurrence of a trouble in the predetermined signal line by performing threshold determination based on a current value of an electric current flowing in the predetermined signal line at predetermined timing in a period for one cycle of the driving signal; and determine an occurrence of a trouble in the predetermined signal line when detecting that the current value continuously deviates predetermined times from a predetermined threshold range.

### BRIEF DESCRIPTION OF THE DRAWINGS

FIG. 1 is a diagram showing a configuration of a main part of a scanning endoscope system according to an embodiment;

FIG. 2 is a sectional view for explaining a configuration of an actuator section;

FIG. 3 is a diagram showing an example of a signal waveform of a driving signal supplied to the actuator section;

FIG. 4 is a diagram showing an example of a spiral scanning route reaching an outermost point B from a center point A;

FIG. 5 is a diagram showing an example of a spiral scanning route reaching the center point A from the outermost point B;

FIG. 6 is a diagram for explaining an example of a temporal change of a current value of an electric current flowing in a signal line connected to the actuator section;

FIG. 7 is a diagram for explaining an example of a temporal change of a current value of an electric current flowing in the signal line connected to the actuator section;

FIG. 8 is a diagram for explaining an example of a temporal change of a current value of an electric current flowing in the signal line connected to the actuator section;

FIG. 9 is a diagram for explaining an example of a configuration usable for determination of presence or absence of occurrence of a trouble in the signal line connected to the actuator section;

FIG. 10 is a diagram for explaining an example of a configuration usable for determination of presence or absence of occurrence of a trouble in the signal line connected to the actuator section;

FIG. 11 is a diagram for explaining an example of a frequency characteristic of a current value of an electric current flowing in the signal line connected to the actuator section;

FIG. 12 is a diagram for explaining an example of a frequency characteristic of a current value of an electric current flowing in the signal line connected to the actuator section; and

FIG. 13 is a diagram for explaining an example of a frequency characteristic of a current value of an electric current flowing in the signal line connected to the actuator section.

### DETAILED DESCRIPTION OF THE PREFERRED EMBODIMENT

An embodiment of the present invention is explained below referring to drawings.

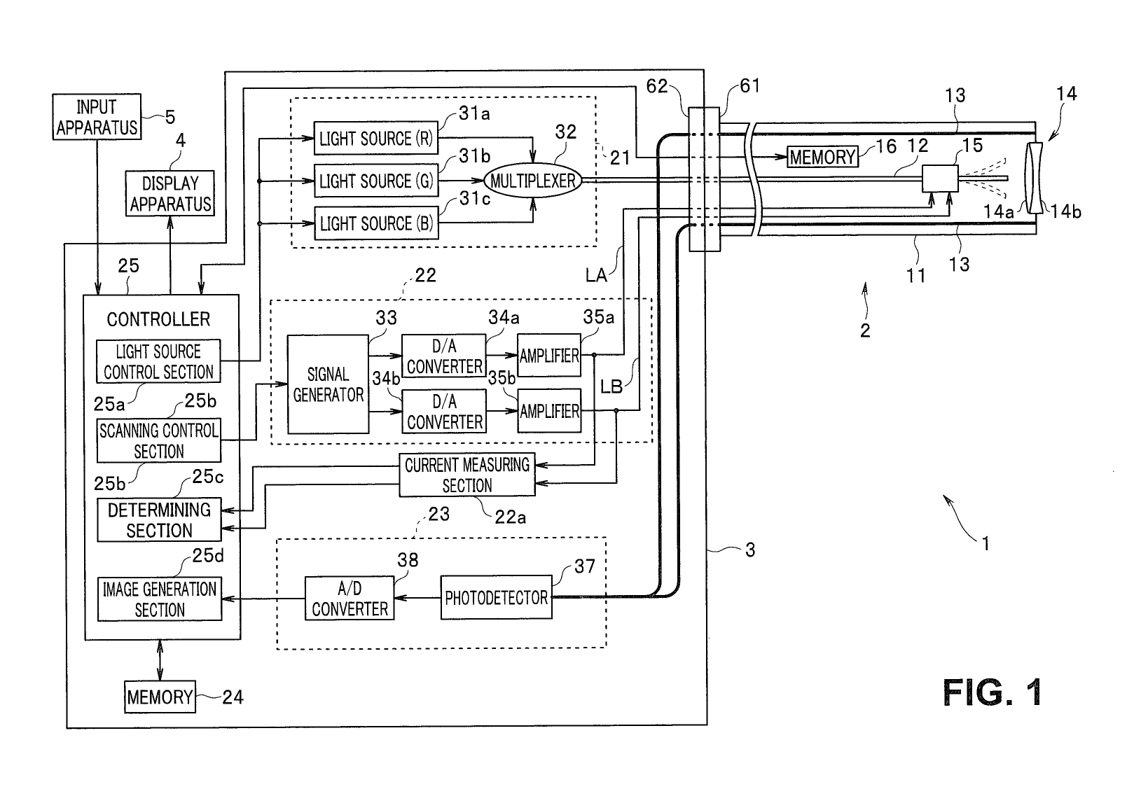

FIG. 1 to FIG. 13 relate to the embodiment of the present invention. FIG. 1 is a diagram showing a configuration of a main part of a scanning endoscope system according to the embodiment.

A scanning endoscope system 1 includes, for example, as shown in FIG. 1, an endoscope 2 of a scanning type to be inserted into a body cavity of a subject, a main body apparatus 3 to which the endoscope 2 is connectable, a display apparatus 4 connected to the main body apparatus 3, and an input apparatus 5 capable of performing an input of information and an instruction to the main body apparatus 3.

The endoscope 2 includes an insertion section 11 formed to have an elongated shape insertable into the body cavity of the subject.

A connector section 61 for detachably connecting the endoscope 2 to a connector receiving section 62 of the main body apparatus 3 is provided in a proximal end portion of the insertion section 11.

Although not shown in FIG. 1, electric connector devices for electrically connecting the endoscope 2 and the main body apparatus 3 are provided inside the connector section 61 and the connector receiving section 62. Although not shown in FIG. 1, optical connector devices for optically connecting the endoscope 2 and the main body apparatus 3 are provided inside the connector section 61 and the connector receiving section 62.

A fiber for illumination 12, which is an optical fiber for guiding illumination light supplied from a light source unit 21 of the main body apparatus 3 and emitting the illumination light from an emission end portion, and a fiber for light reception 13 including one or more optical fibers for receiving return light from an object and guiding the return light to a detecting unit 23 of the main body apparatus 3 are respectively inserted through a portion extending from a proximal end portion to a distal end portion inside the insertion section 11.

An incident end portion including a light incident surface of the fiber for illumination 12 is disposed in a multiplexer 32 provided inside the main body apparatus 3. An emission end portion including a light emission surface of the fiber for illumination 12 is disposed near a light incident surface of a lens 14a provided at a distal end portion of the insertion section 11.

An incident end portion including a light incident surface of the fiber for light reception 13 is fixed and disposed around a light emission surface of a lens 14b on a distal end face of the distal end portion of the insertion section 11. An emission end portion including a light emission surface of the fiber for light reception 13 is disposed in a photodetector 37 provided inside the main body apparatus 3.

An illumination optical system 14 is provided at the distal end portion of the insertion section 11. The illumination optical system 14 includes the lens 14a on which the illumination light passed through the light emission surface of the fiber for illumination 12 is made incident and the lens 14b configured to emit the illumination light passed through the lens 14a to the object.

In a halfway portion of the fiber for illumination 12 on the distal end portion side of the insertion section 11, an actuator section 15 configured to be driven based on a driving signal supplied from a driver unit 22 of the main body apparatus 3 is provided.

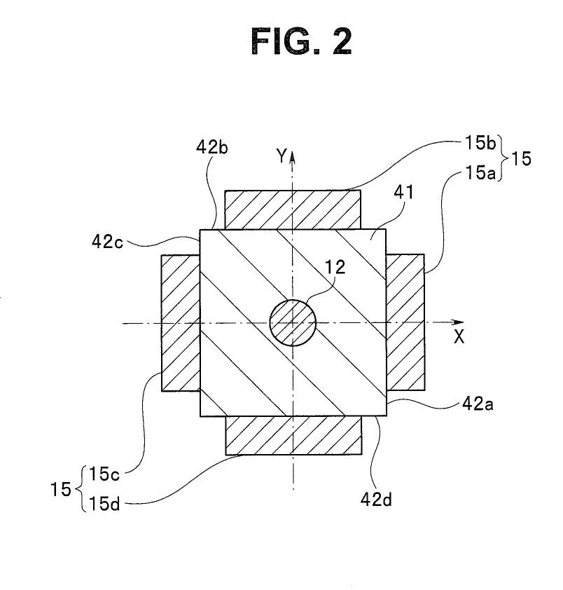

The fiber for illumination 12 and the actuator section 15 are respectively disposed to have, for example, a positional relation shown in FIG. 2 on a cross section perpendicular to a longitudinal axis direction of the insertion section 11. FIG. 2 is a sectional view for explaining a configuration of the actuator section.

As shown in FIG. 2, a ferrule 41 functioning as a joining member is disposed between the fiber for illumination 12 and the actuator section 15. More specifically, the ferrule 41 is formed by, for example, zirconium (ceramic), nickel, or the like.

As shown in FIG. 2, the ferrule 41 is formed in a square pole. The ferrule 41 includes side surfaces 42a and 42c perpendicular to an X-axis direction, which is a first axial direction orthogonal to the longitudinal axis direction of the insertion section 11, and side surfaces 42b and 42d perpendicular to a Y-axis direction, which is a second axial direction orthogonal to the longitudinal axis direction of the insertion section 11. The fiber for illumination 12 is fixed and disposed in a center of the ferrule 41. Note that the ferrule 41 may be formed in another shape other than the square pole as long as the ferrule 41 has a columnar shape.

For example, as shown in FIG. 2, the actuator section 15 includes a piezoelectric element 15a disposed along the side surface 42a, a piezoelectric element 15b disposed along the side surface 42b, a piezoelectric element 15c disposed along the side surface 42c, and a piezoelectric element 15d disposed along the side surface 42d.

The piezoelectric elements 15a to 15d have polarization directions individually set in advance. The piezoelectric elements 15a to 15d are configured to respectively expand and contract according to a driving voltage applied by a driving signal supplied from the main body apparatus 3.

That is, the piezoelectric elements 15a and 15c of the actuator section 15 are configured as an actuator for X axis capable of swinging the fiber for illumination 12 in the X-axis direction by vibrating according to the driving signal supplied from the main body apparatus 3. The piezoelectric elements 15b and 15d of the actuator section 15 are configured as an actuator for Y axis capable of swinging the fiber for illumination 12 in the Y-axis direction by vibrating according to the driving signal supplied from the main body apparatus 3.

A nonvolatile memory 16 for storing endoscope information, which is information peculiar to each endoscope 2, is provided inside the insertion section 11. The endoscope information stored in the memory 16 is read out by a controller 25 as a hardware device of the main body apparatus 3 when the connector section 61 of the endoscope 2 and the connector receiving section 62 of the main body apparatus 3 are connected and a power supply of the main body apparatus 3 is turned on.

The main body apparatus 3 includes the light source unit 21, the driver unit 22, a current measuring section 22a, the detecting unit 23, a memory 24, and the controller 25.

The light source unit 21 includes a light source 31a, a light source 31b, a light source 31c, and the multiplexer 32.

The light source 31a includes, for example, a laser light source configured to emit light in a wavelength band of red (hereinafter referred to as R light as well). The light source 31a is configured to be switched to a light emitting state (an ON state) or an extinguished state (an OFF state) according to control by the controller 25. The light source 31a is configured to emit the R light having a light amount corresponding to the control by the controller 25 in the light emitting state.

The light source 31b includes, for example, a laser light source configured to emit light in a wavelength band of green (hereinafter referred to as G light). The light source 31b is configured to be switched to a light emitting state (an ON state) or an extinguished state (an OFF state) according to the control by the controller 25. The light source 31b is configured to emit the G light having a light amount corresponding to the control by the controller 25 in the light emitting state.

The light source 31c includes, for example, a laser light source configured to emit light in a wavelength band of blue (hereinafter referred to as B light as well). The light source 31c is configured to be switched to a light emitting state (an ON state) or an extinguished state (an OFF state) according to the control by the controller 25. The light source 31c is configured to emit the B light having a light amount corresponding to the control by the controller 25 in the light emitting state.

The multiplexer 32 is configured to be capable of multiplexing the R light emitted from the light source 31a, the G light emitted from the light source 31b, and the B light emitted from the light source 31c and supplying the multiplexed light to the light incident surface of the fiber for illumination 12.

The driver unit 22 is configured to be electrically connected to the actuator section 15 via signal lines LA and LB when the connector section 61 and the connector receiving section 62 are connected. The driver unit 22 is configured to generate, based on the control by the controller 25, a driving signal DA having periodicity as a signal for driving an actuator for X axis of the actuator section 15 and supply the generated driving signal DA to the piezoelectric elements 15a and 15c via the signal line LA connected to the actuator section 15. The driver unit 22 is configured to generate, based on the control by the controller 25, a driving signal DB having periodicity as a signal for driving the actuator for Y axis of the actuator section 15 and supply the generated driving signal DB to the piezoelectric elements 15b and 15d via a signal line LB connected to the actuator section 15. That is, the driver unit 22 includes a function of a driving-signal supplying section. The driver unit 22 includes a signal generator 33, D/A converters 34a and 34b, and amplifiers 35a and 35b.

The signal generator 33 is configured to generate, based on the control by the controller 25, as a first driving control signal for swinging the emission end portion of the fiber for illumination 12 in the X-axis direction, for example, a signal having a waveform indicated by equation (1) described below and output the signal to the D/A converter 34a. Note that, in equation (1) described below, X(t) represents a signal level at time t, Ax represents an amplitude value not depending on the time t, and G(t) represents a predetermined function used for modulation of a sine wave sin(2πft).

X(t)=Ax×G(t)×sin(2πft) (1)

The signal generator 33 is configured to generate, based on the control by the controller 25, as a second driving control signal for swinging the emission end portion of the fiber for illumination 12 in the Y-axis direction, for example, a signal having a waveform indicated by equation (2) described below and output the signal to the D/A converter 34b. Note that, in equation (2) described below, Y(t) represents a signal level at the time t, Ay represents an amplitude value not depending on the time t, G(t) represents a predetermined function used for modulation of a sine wave sin(2πft+φ), and φ represents a phase.

Y(t)=Ay×G(t)×sin(2πft+φ) (2)

The D/A converter 34a is configured to convert a digital first driving control signal outputted from the signal generator 33 into the driving signal DA, which is an analog voltage signal, and output the driving signal DA to the amplifier 35a.

The D/A converter 34b is configured to convert a digital second driving control signal outputted from the signal generator 33 into the driving signal DB, which is an analog voltage signal, and output the driving signal DB to the amplifier 35b.

The amplifier 35a includes, for example, a signal amplifier circuit. The amplifier 35a is configured to be electrically connected to the piezoelectric elements 15a and 15c of the actuator section 15 via the signal line LA when the connector section 61 and the connector receiving section 62 are connected. The amplifier 35a is configured to amplify the driving signal DA outputted from the D/A converter 34a and output the amplified driving signal DA to the piezoelectric elements 15a and 15c via the signal line LA.

The amplifier 35b includes, for example, a signal amplification circuit. The amplifier 35b is configured to be electrically connected to the piezoelectric elements 15b and 15d of the actuator section 15 via the signal line LB when the connector section 61 and the connector receiving section 62 are connected. The amplifier 35b is configured to amplify the driving signal DB outputted from the D/A converter 34b and output the amplified driving signal DB to the piezoelectric elements 15b and 15d via the signal line LB.

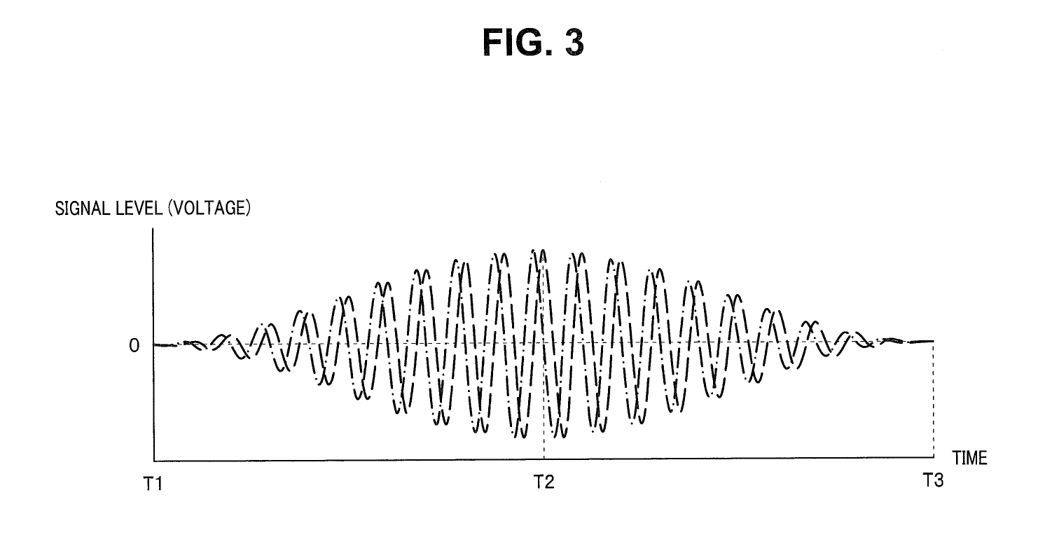

For example, when Ax=Ay and φ=π/2 are set in equations (1) and (2) described above, a driving voltage corresponding to the driving signal DA having a signal waveform indicated by a broken line shown in FIG. 3, that is, a signal waveform having a period from time T1 to time T3 as a period for one cycle is applied to the piezoelectric elements 15a and 15c of the actuator section 15. A driving voltage corresponding to the driving signal DB having a signal waveform indicated by an alternate long and short dash line shown in FIG. 3, that is, a signal waveform having the period from the time T1 to the time T3 as a period for one cycle is applied to the piezoelectric elements 15b and 15d of the actuator section 15. FIG. 3 is a diagram showing an example of a signal waveform of a driving signal supplied to the actuator section.

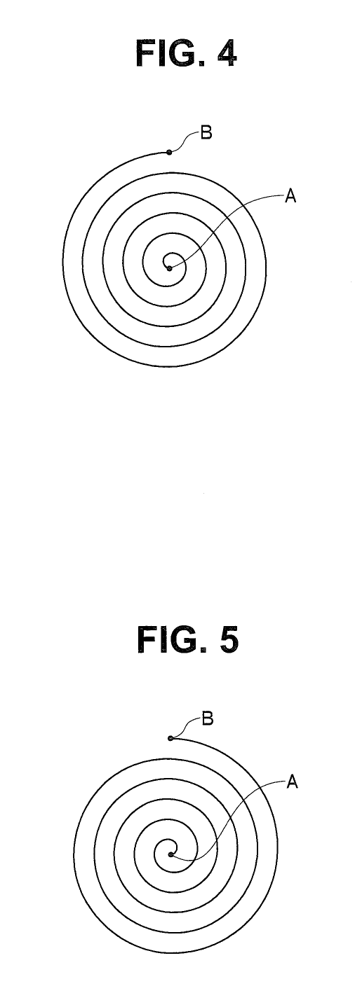

For example, when the driving voltage corresponding to the driving signal DA having the signal waveform indicated by the broken line shown in FIG. 3 is applied to the piezoelectric elements 15a and 15c of the actuator section 15 and the driving voltage corresponding to the driving signal DB having the signal waveform indicated by the alternate long and short dash line shown in FIG. 3 is applied to the piezoelectric elements 15b and 15d of the actuator section 15, the emission end portion of the fiber for illumination 12 is spirally swung. A surface of the object is scanned in a spiral scanning route shown in FIG. 4 and FIG. 5 according to such a swing. FIG. 4 is a diagram showing an example of a spiral scanning route reaching an outermost point B from a center point A. FIG. 5 is a diagram showing an example of a spiral scanning route reaching the center point A from the outermost point B.

More specifically, first, at the time T1, illumination light is irradiated on a position equivalent to the center point A of an irradiation position of the illumination light on the surface of the object. Thereafter, as signal levels (voltages) of the driving signals DA and DB increase from the time T1 to the time T2, the irradiation position of the illumination light on the surface of the object is displaced to draw a first spiral scanning route to an outer side starting from the center point A. Further, when time reaches the time T2, the illumination light is irradiated on the outermost point B of the irradiation position of the illumination light on the surface of the object. As the signal levels (the voltages) of the driving signals DA and DB decrease from the time T2 to the time T3, the irradiation position of the illumination light on the surface of the object is displaced to draw a second spiral scanning route to an inner side starting from the outermost point B. Further, when the time reaches the time T3, the illumination light is irradiated on the center point A on the surface of the object.

That is, the actuator section 15 includes a configuration capable of displacing, along the spiral scanning routes shown in FIG. 4 and FIG. 5, the irradiation position of the illumination light emitted to the object through the emission end portion by swinging the emission end portion of the fiber for illumination 12 based on the driving signals DA and DB supplied from the driver unit 22.

The current measuring section 22a is configured to measure a current value IA of the driving signal DA supplied to the piezoelectric elements 15a and 15c of the actuator section 15 via the signal line LA and output the measured current value IA to the controller 25. The current measuring section 22a is configured to measure a current value IB of the driving signal DB supplied to the piezoelectric elements 15b and 15d of the actuator section 15 via the signal line LB and output the measured current value IB to the controller 25.

The detecting unit 23 is configured to detect a return light received by the fiber for light reception 13 of the endoscope 2 and generate and output a light detection signal corresponding to intensity of the detected return light. More specifically, the detecting unit 23 includes the photodetector 37 and an A/D converter 38.

The photodetector 37 includes, for example, an avalanche photodiode. The photodetector 37 is configured to detect light (return light) emitted from the light emission surface of the fiber for light reception 13, generate an analog light detection signal corresponding to intensity of the detected light, and sequentially output the light detection signal to the A/D converter 38.

The A/D converter 38 is configured to convert the analog light detection signal outputted from the photodetector 37 into a digital light detection signal and sequentially output the digital light detection signal to the controller 25.

In the memory 24, control information used in control of the main body apparatus 3 is stored. More specifically, in the memory 24, as the control information used in the control of the main body apparatus 3, for example, information including parameters such as a frequency for specifying a signal waveform shown in FIG. 3 and a mapping table used for generation of an observation image to be displayed on the display apparatus 4 is stored. Note that the mapping table described above may be configured in, for example, a format capable of specifying a correspondence relation between output timing of a light detection signal sequentially outputted from the detecting unit 23 and a pixel position to which pixel information obtained by converting the light detection signal is applied.

Current thresholds THA and THB to be used in determination by a determining section 25c explained below are also stored in the memory 24.

The controller 25 is configured of an integrated circuit such as an FPGA (field programmable gate array). The controller 25 is configured to be capable of detecting whether the insertion section 11 is electrically connected to the main body apparatus 3 by detecting a connection state of the connector section 61 in the connector receiving section 62 via a not-shown signal line or the like. The controller 25 is configured to read endoscope information from the memory 16 when the connector section 61 and the connector receiving section 62 are connected and the power supply of the main body apparatus 3 is turned on. The controller 25 is configured to read the control information and the current thresholds THA and THB from the memory 24 when the power supply of the main body apparatus 3 is turned on. The controller 25 includes a light-source control section 25a, a scanning control section 25b, a determining section 25c, and an image generating section 25d.

The light-source control section 25a is configured to be capable of performing operation for individually switching the respective light sources of the light source unit 21 to the ON state or the OFF state. The light-source control section 25a is configured to be capable of individually adjusting light amounts of the R light, the G light, and the B light emitted from the respective light sources of the light source unit 21. The light-source control section 25a is configured to perform, on the light source unit 21, based on the control information read from the memory 24, for example, control for causing the light source unit 21 to repeatedly emit the R light, the G light, and the B light in this order. The light-source control section 25a is configured to perform, when detecting that a predetermined determination result is obtained by the determination of the determining section 25c, control for causing the light source unit 21 to stop the supply of the R light, the G light, and the B light from the light source unit 21.

The scanning control section 25b is configured to perform, on the driver unit 22, based on the control information read from the memory 24, for example, control for causing the driver unit 22 to generate the driving signals DA and DB having the signal waveform shown in FIG. 3. The scanning control section 25b is configured to perform, when detecting that the predetermined determination result is obtained by the determination of the determining section 25c, control for causing the driver unit 22 to stop the supply of the driving signals DA and the DB from the driver unit 22.

The determining section 25c is configured to determine, based on the current thresholds THA and THB read from the memory 24 and the current values IA and IB outputted from the current measuring section 22a, presence or absence of occurrence of a trouble in the signal lines LA and LB. Note that a specific example of a determining method for presence or absence of occurrence of a trouble in the signal lines LA and LB is explained below.

The image generating section 25d is configured to convert, based on the mapping table included in the control information read from the memory 24, for example, light detection signals sequentially outputted from the detecting unit 23 in the period from the time T1 to the time T2 into pixel information such as RGB components and map (arrange) the pixel information to thereby generate an observation image frame by frame and sequentially output the generated observation image to the display apparatus 4.

The display apparatus 4 includes, for example, an LCD (liquid crystal display). The display apparatus 4 is configured to be capable of displaying the observation image outputted from the main body apparatus 3.

The input apparatus 5 includes, for example, a keyboard or a touch panel. Note that the input apparatus 5 may be configured as an apparatus separate from the main body apparatus 3 or may be configured as an interface integrated with the main body apparatus 3.

Subsequently, operation and the like of the scanning endoscope system 1 including the configuration explained above are explained.

After connecting the respective sections of the scanning endoscope system 1 and turning on the power supply, for example, a user such as a surgeon turns on a scanning start switch (not shown in FIG. 1) of the input apparatus 5 to thereby give, to the controller 25, an instruction for causing the controller 25 to start scanning of a desired object by the endoscope 2.

When the scanning start switch of the input apparatus 5 is turned on, the light-source control section 25a performs, on the light source unit 21, control for causing the light source unit 21 to repeatedly emit the R light, the G light, and the B light in this order.

When the scanning start switch of the input apparatus 5 is turned on, the scanning control section 25b performs, on the driver unit 22, control for causing the driver unit 22 to generate the driving signals DA and DB having the signal waveform shown in FIG. 3. According to such control by the scanning control section 25b, the driving signal DA is supplied to the piezoelectric elements 15a and 15c via the signal line LA. The current value IA of an electric current flowing in the signal line LA caused by the supply of the driving signal DA is measured by the current measuring section 22a. The measured current value IA is sequentially outputted from the current measuring section 22a to the determining section 25c. According to the control by the scanning control section 25b explained above, the driving signal DB is supplied to the piezoelectric elements 15b and 15d via the signal line LB. The current value IB of an electric current flowing in the signal line LB caused by the supply of the driving signal DB is measured by the current measuring section 22a. The measured current value IB is sequentially outputted from the current measuring section 22a to the determining section 25c.

The determining section 25c determines, based on the current thresholds THA and THB and the current values IA and IB outputted from the current measuring section 22a, presence or absence of occurrence of a trouble in the signal lines LA and LB.

A specific example and the like of a determining method for presence or absence of occurrence of a trouble in the signal lines LA and LB are explained here. Note that, according to the embodiment, presence or absence of occurrence of a trouble in the signal line LA and presence or absence of occurrence of a trouble in the signal line LB are determined individually and by a common determining method. Therefore, in the following explanation, a method for determining presence or absence of occurrence of a trouble in the signal line LA is mainly explained. On the other hand, a method for determining presence or absence of occurrence of a trouble in the signal line LB is simplified as appropriate and explained.

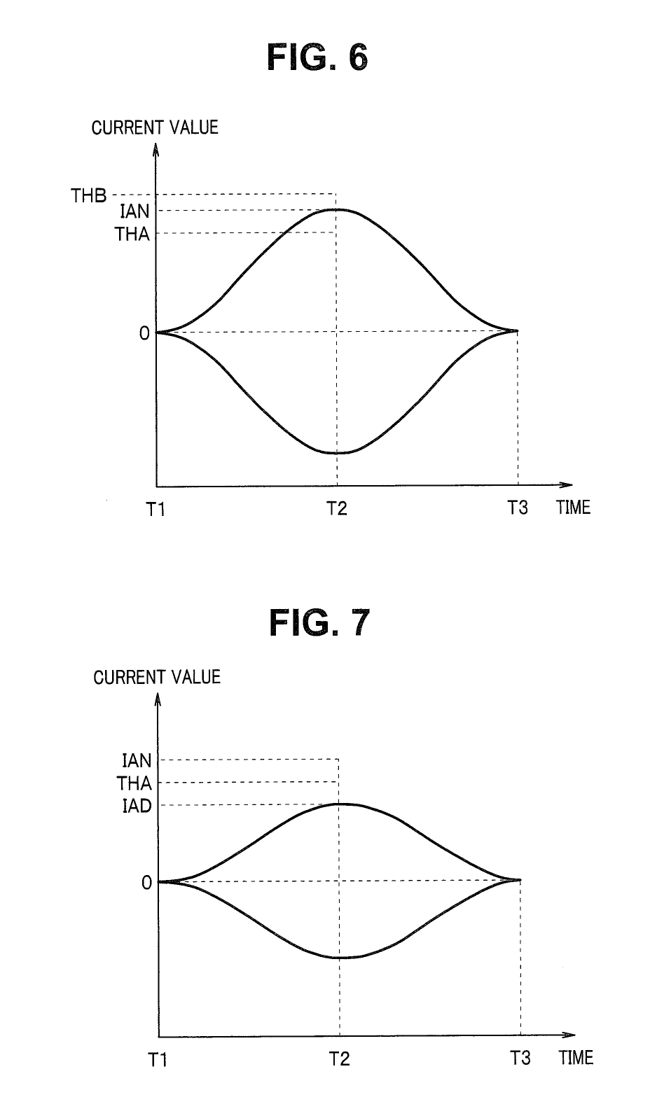

According to an experiment result of an applicant, it is confirmed that, when the signal line LA is normal, a temporal change of the current value IA in a period for one cycle from the time T1 to the time T3 is observed as, for example, an envelope waveform centering on IA=0 shown in FIG. 6. According to an experiment result of the applicant, it is confirmed that, when the signal line LA is normal, as illustrated in FIG. 6, the current value IA reaches a maximum current value IAN at timing of the time T2. FIG. 6 is a diagram for explaining an example of a temporal change of a current value of an electric current flowing in the signal line connected to the actuator section.

According to an experiment result of the applicant, it is confirmed that, when disconnection has occurred in the signal line LA, a temporal change of the current value IA in the period for one cycle from the time T1 to the time T3 is observed as, for example, an envelope waveform centering on IA=0 shown in FIG. 7. According to an experiment result of the applicant, it is confirmed that, when disconnection has occurred in the signal line LA, a phenomenon occurs in which the current value IA measured at the timing of the time T2 decreases to a maximum current value IAD, which is a value smaller than the maximum current value IAN at normal times (see FIG. 7). FIG. 7 is a diagram for explaining an example of a temporal change of a current value of an electric current flowing in the signal line connected to the actuator section. Note that, according to an experiment result of the applicant, it is confirmed that an envelope waveform substantially the same as the envelope waveform shown in FIG. 7 is observed when the signal lines LA and LB in a live-line state (during the supply of the driving signals DA and DB to the actuator section 15) come into contact.

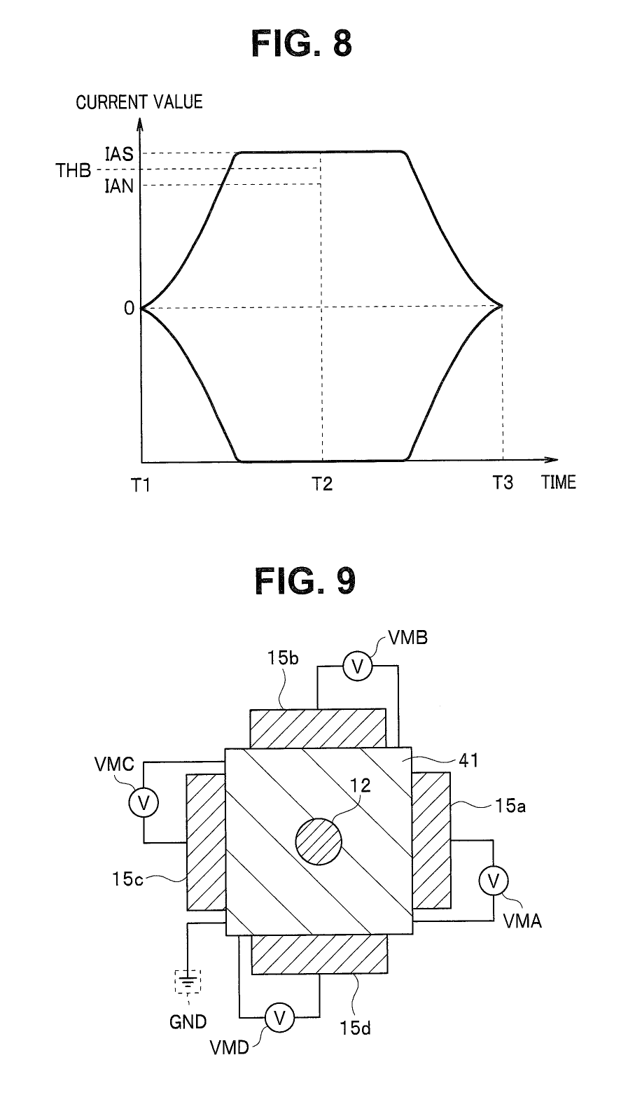

According to an experiment result of the applicant, it is confirmed that, when a short circuit has occurred in the signal line LA, a temporal change of the current value IA in the period for one cycle from the time T1 to the time T3 is observed as, for example, an envelope waveform centering on IA=0 shown in FIG. 8. According to an experiment result of the applicant, it is confirmed that, when a short circuit has occurred in the signal line LA, a phenomenon occurs in which the current value IA measured in a fixed period including the time T2 in the period for one cycle from the time T1 to the time T3 is maintained at a maximum current value IAS, which is a value larger than the maximum current value IAN at normal times (see FIG. 8). FIG. 8 is a diagram for explaining an example of a temporal change of a current value of an electric current flowing in the signal line connected to the actuator section.

Note that knowledge obtained from the respective experiment results enumerated above is not limited to be applied to only the current value IA of the electric current flowing in the signal line LA caused by the supply of the driving signal DA. The knowledge is applied substantially in the same manner to the current value IB flowing in the signal line LB caused by the supply of the driving signal DB.

In view of the knowledge obtained from the respective experiment results enumerated above, in the embodiment, the determining section 25c determines presence or absence of occurrence of a trouble in the signal line LA based on whether a current value IA2 equivalent to the current value IA outputted from the current measuring section 22a at the timing of the time T2 is within a threshold range between the current threshold THA (see FIG. 6 and FIG. 7) set to a value smaller than the maximum current value IAN and the current threshold THB (see FIG. 6 and FIG. 8) set to a value larger than the maximum current value IAN. In the embodiment, the determining section 25c determines presence or absence of occurrence of a trouble in the signal line LB based on whether a current value IB2 equivalent to the current value IB outputted from the current measuring section 22a at the timing of the time T2 is within the threshold range between the current threshold THA and the current threshold THB.

More specifically, when detecting that the current value IA2 smaller than the current threshold THA, which is a lower limit value of the threshold range, is continuously outputted from the current measuring section 22a P (2≤P) times, the determining section 25c obtains a determination result that disconnection has occurred in the signal line LA (or the signal lines LA and LB are in contact). When detecting that the current value IA2 larger than the current threshold THB, which is an upper limit value of the threshold range, is continuously outputted P times from the current measuring section 22a, the determining section 25c obtains a determination result that a short circuit has occurred in the signal line LA. When detecting that the number of times the current value IA2 smaller than the current threshold THA is continuously outputted from the current measuring section 22a is smaller than P times or the number of times the current value IA2 larger than the current threshold THB is continuously outputted from the current measuring section 22a is smaller than P times, the determining section 25c obtains a determination result that a trouble has not occurred in the signal line LA.

That is, when detecting that the current value IA2 outputted from the current measuring section 22a has continuously deviated P times from a threshold range of the current threshold THA or more and the current threshold THB or less, the determining section 25c obtains a determination result that a trouble has occurred in the signal line LA. When detecting that the number of times the current value IA2outputted from the current measuring section 22a has continuously deviated from the threshold range of the current threshold THA or more and the current threshold THB or less has not reached P times, the determining section 25c obtains a determination result that a trouble has not occurred in the signal line LA.

When detecting that the current value IB2 smaller than the current threshold THA is continuously outputted P times from the current measuring section 22a, the determining section 25c obtains a determination result that disconnection has occurred in the signal line LB (or the signal lines LA and LB are in contact). When detecting that the current value IB2 larger than the current threshold THB is continuously outputted P times from the current measuring section 22a, the determining section 25c obtains a determination result that a short circuit has occurred in the signal line LB. When detecting that the number of times the current value IB2 smaller than the current threshold THA is continuously outputted from the current measuring section 22a is smaller than P times or when detecting that the number of times the current value IB2 larger than the current threshold THB is continuously outputted from the current measuring section 22a is smaller than P times, the determining section 25c obtains a determination result that a trouble has not occurred in the signal line LB.

That is, when detecting that the current value IB2 outputted from the current measuring section 22a has continuously deviated P times from the threshold range of the current threshold THA or more and the current threshold THB or less, the determining section 25c obtains a determination result that a trouble has occurred in the signal line LB. When the number of times the current value IB2 outputted from the current measuring section 22a has continuously deviated from the threshold range of the current threshold THA or more and the current threshold THB or less has not reached P times, the determining section 25c obtains a determination result that a trouble has not occurred in the signal line LB.

Note that, in the embodiment, it is desirable that the current threshold THA used for the determination of the determining section 25c is set to, for example, approximately an intermediate value between the maximum current value IAN and the maximum current value IAD.

In the embodiment, it is desirable that the current threshold THB used for the determination of the determining section 25c is set to, for example, approximately an intermediate value between the maximum current value IAN and the maximum current value IAS.

In the embodiment, it is desirable that a value of P used for the determination of the determining section 25c is set to, for example, a value so that an instantaneous change of the current value IA (or IB) that occurs because of a disturbance or the like to the insertion section 11 can be distinguished from a permanent change of the current value IA (or IB) that occurs because of a trouble of the signal line LA (or LB). More specifically, in the embodiment, it is desirable that P is set to a value of approximately 5.

When detecting that the determination result that a trouble has occurred in at least one of the signal lines LA and LB is obtained by the determination of the determining section 25c, the light-source control section 25a performs control for stopping the supply of the R light, the G light, and the B light from the light source unit 21 while invalidating an instruction corresponding to operation of the scanning start switch of the input apparatus 5. On the other hand, when detecting that the determination result that a trouble has not occurred in either of the signal lines LA or LB is obtained by the determination of the determining section 25c, the light-source control section 25a continues the control for causing the light source unit 21 to repeatedly emit the R light, the G light, and the B light in this order.

When detecting that the determination result that a trouble has occurred in at least one of the signal lines LA and LB is obtained by the determination of the determining section 25c, the scanning control section 25b performs control for stopping the supply of the driving signals DA and DB from the driver unit 22 while invalidating an instruction corresponding to operation of the scanning start switch of the input apparatus 5. On the other hand, when detecting that the determination result that a trouble has not occurred in either of the signal lines LA or LB is obtained by the determination of the determining section 25c, the scanning control section 25b continues the control for causing the driver unit 22 to generate the driving signals DA and DB having the signal waveform shown in FIG. 3.

As explained above, according to the embodiment, it is possible to detect, based on the current values IA2 and IB2 outputted from the current measuring section 22a at every timing of the time T2, occurrence of a trouble in at least one of the signal lines LA and LB connected to the actuator section 15. Therefore, according to the embodiment, it is possible to surely detect occurrence of a trouble of a signal line connected to an actuator for optical scanning.

Note that, according to the embodiment, the determining section 25c is not limited to determining presence or absence of occurrence of a trouble in the signal lines LA and LB based on the current values IA and IB measured by the current measuring section 22a. The determining section 25c may determine presence or absence of occurrence of a trouble in the signal lines LA and LB based on a voltage value of a voltage applied to the piezoelectric elements 15a to 15d.

More specifically, for example, when the ferrule 41 is connected to a GND (ground potential) as shown in FIG. 9, the determining section 25c may determine presence or absence of occurrence of a trouble in the signal line LA based on whether at least one of a voltage value VA2 measured at every timing of the time T2 by a voltmeter VMA connected to the ferrule 41 and the piezoelectric element 15a and a voltage value VC2 measured at every timing of the time T2 with a voltmeter VMC connected to the ferrule 41 and the piezoelectric element 15c is continuously smaller than a predetermined threshold (of a voltage value) P times. For example, when the ferrule 41 is connected to the GND as shown in FIG. 9, the determining section 25c may determine presence or absence of occurrence of a trouble in the signal line LB based on whether at least one of a voltage value VB2 measured at every timing of the time T2 by a voltmeter VMB connected to the ferrule 41 and the piezoelectric element 15b and a voltage value VD2 measured at every timing of the time T2 with a voltmeter VMD connected to the ferrule 41 and the piezoelectric element 15d is continuously smaller than the predetermined threshold (of a voltage value) P times. FIG. 9 is a diagram for explaining an example of a configuration usable for determination of presence or absence of occurrence of a trouble in the signal line connected to the actuator section.

According to the determination of the determining section 25c explained above, for example, when it is detected that a voltage value VA2 smaller than the predetermined threshold (of a voltage value) is continuously outputted P times from the voltmeter VMA, a determination result that a trouble such as disconnection or a short circuit has occurred in the signal line LA is obtained. According to the determination of the determining section 25c explained above, for example, when it is detected that the voltage value VB2 smaller than the predetermined threshold (of a voltage value) is continuously outputted P times from the voltmeter VMB, a determination result that a trouble such as disconnection or a short circuit has occurred in the signal line LB is obtained.

According to the embodiment, the voltage values VA to VD are not limited to be simultaneously measured using the four voltmeters VMA to VMD. For example, the voltage values VA to VD may be measured in order using one voltmeter. According to the embodiment, the voltmeters VMA to VMD may be provided in either the endoscope 2 or the main body apparatus 3.

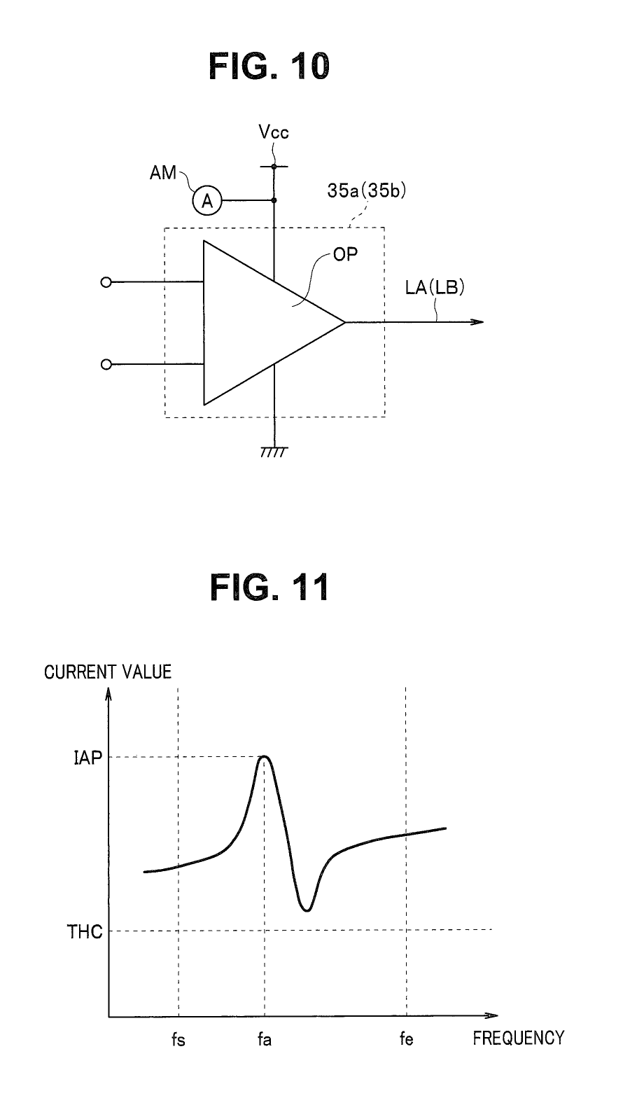

According to the embodiment, for example, as shown in FIG. 10, the amplifier 35a (the amplifier 35b) may include an operational amplifier OP connected to the actuator section 15 via the signal line LA (the signal line LB) and configured to amplify the driving signal DA (the driving signal DB) outputted from the D/A converter 34a (the D/A converter 34b) and supply the driving signal DA (the driving signal DB) to the actuator section 15. When the amplifier 35a (the amplifier 35b) includes the operational amplifier OP, the determining section 25c may determine presence or absence of occurrence of a trouble in the signal line LA (the signal line LB) based on a current value measured by an ammeter AM connected to a power supply line for supplying a power supply voltage Vcc to the operational amplifier OP, that is, a current value of an electric current flowing in the power supply line. With such a determining method, for example, when detecting based on the current value measured by the ammeter AM that a large current flows to the operational amplifier OP, the determining section 25c can obtain a determination result that a short circuit has occurred in the signal line LA (the signal line LB). Therefore, it is possible to quickly stop the supply of the R light, the G light, and the B light from the light source unit 21 and the supply of the driving signals DA and DB from the driver unit 22. FIG. 10 is a diagram for explaining an example of a configuration usable for determination of presence or absence of occurrence of a trouble in the signal line connected to the actuator section.

According to the embodiment, for example, when a determination result that a trouble has occurred in at least one of the signal lines LA and LB is obtained, operation for notifying the determination result to the user with a character string or the like may be performed in the controller 25.

On the other hand, by modifying the configurations of the respective sections of the embodiment as appropriate, the determining section 25c may determine presence or absence of occurrence of a trouble in the endoscope 2 based on, for example, a frequency characteristic of a current value of an electric current flowing in the signal line LA obtained by sweeping, within a predetermined range from a lower limit frequency fs to an upper limit frequency fe, a frequency in supplying a driving signal DC having a constant signal level, such as a sine wave, to the actuator section 15. When a determination result that a trouble has occurred in the endoscope 2 is obtained by such determination of the determining section 25c, operation for causing the display apparatus 4 to display a character string or the like for notifying the determination result to the user may be performed in the controller 25.

With the configuration according to the modification explained above, for example, when a frequency characteristic shown in FIG. 11 is obtained, that is, a maximum current value (a peak current value) IAP is measured at a frequency fa, which satisfies fs<fa<fe, and a frequency characteristic that the measured maximum current value IAP is larger than a current threshold THC is obtained, a determination result that a trouble has not occurred in the endoscope 2 is obtained. FIG. 11 is a diagram for explaining an example of a frequency characteristic of a current value of an electric current flowing in the signal line connected to the actuator section

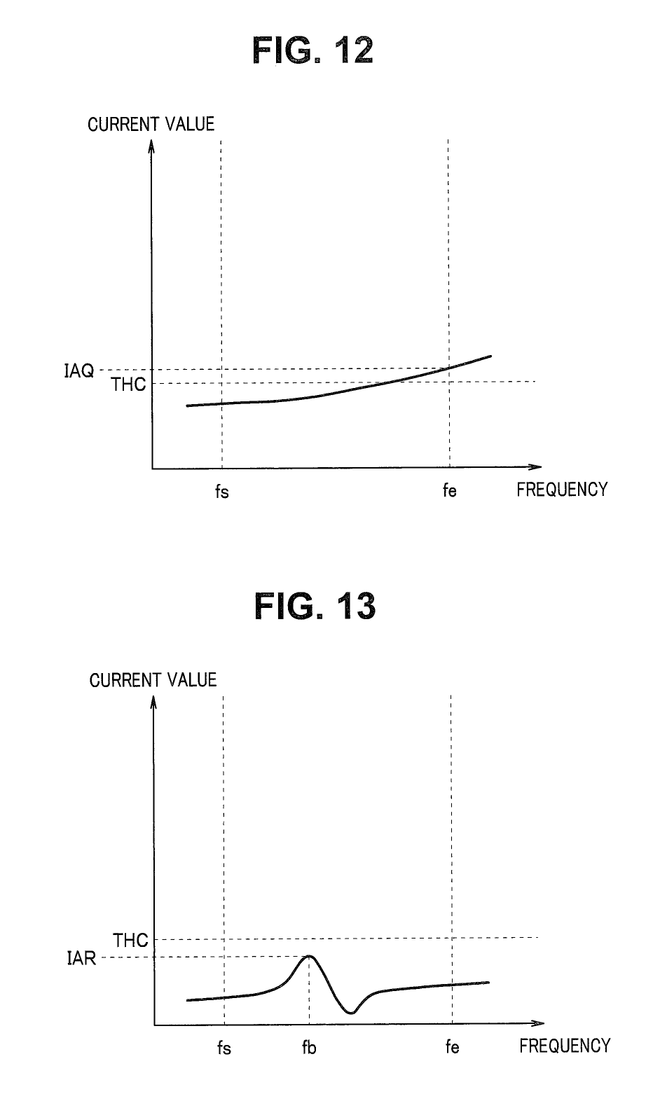

With the configuration according to the modification explained above, for example, when a frequency characteristic shown in FIG. 12 is obtained, that is, when a maximum current value IAQ is measured at an upper limit frequency fe and a frequency characteristic that the measured maximum current value IAQ is larger than the current threshold THC is obtained, a determination result that a trouble has occurred in either the piezoelectric element 15a or 15c is obtained. FIG. 12 is a diagram for explaining an example of a frequency characteristic of a current value of an electric current flowing in the signal line connected to the actuator section.

With the configuration according to the modification explained above, for example, when a frequency characteristic shown in FIG. 13 is obtained, that is, when a maximum current value IAR is measured at a frequency fb which satisfies fs<fb<fe, and a frequency characteristic that the measured maximum current value IAR is equal to or smaller than the current threshold THC is obtained, a determination result that a trouble has occurred in either the signal line LA or a wire of the GND is obtained. FIG. 13 is a diagram for explaining an example of a frequency characteristic of a current value of an electric current flowing in the signal line connected to the actuator section.

Note that, by changing a part of the modification explained above as appropriate, the determining section 25c may determine presence or absence of occurrence of a trouble in the endoscope 2 based on a frequency characteristic of a current value of an electric current flowing in the signal line LB.

The present invention is not limited to the embodiment and the modification explained above. It goes without saying that various changes and applications are possible within a range not departing from the spirit of the invention.

What is claimed is:

1. A scanning endoscope system comprising:
an optical fiber for guiding illumination light supplied from a light source section;
an actuator configured to displace an irradiation position of the illumination light emitted from the optical fiber; and
a controller configured to:
generate a driving signal including periodicity as a signal for driving the actuator,
supply the driving signal to the actuator via a predetermined signal line connected to the actuator,
detect presence or absence of occurrence of a trouble in the predetermined signal line by performing threshold determination based on a current value of an electric current flowing in the predetermined signal line at predetermined timing in a period for one cycle of the driving signal, and
determine an occurrence of a trouble in the predetermined signal line when detecting that the current value continuously deviates predetermined times from a predetermined threshold range.

2. The scanning endoscope system according to claim 1,
wherein the controller determines an occurrence of a trouble as disconnection in the predetermined signal line when detecting that the current value is continuously smaller the predetermined times than a lower limit value of the predetermined threshold range.

3. The scanning endoscope system according to claim 1,
wherein the controller determines an occurrence of a trouble as short circuit in the predetermined signal line when detecting that the current value is continuously larger the predetermined times than an upper limit value of the predetermined threshold range.

4. A scanning endoscope system comprising:
an optical fiber for guiding illumination light supplied from a light source section;
an actuator configured to displace an irradiation position of the illumination light emitted from the optical fiber; and
a controller configured to:
generate a driving signal including periodicity as a signal for driving the actuator,
supply the driving signal to the actuator via a predetermined signal line connected to the actuator,
detect presence or absence of occurrence of a trouble in the predetermined signal line by performing threshold determination based on a voltage value of a voltage applied to the actuator at the predetermined timing in a period for one cycle of the driving signal, and
determine an occurrence of a trouble in the predetermined signal line when detecting that the voltage value is continuously lower predetermined times than a predetermined threshold.

5. A scanning endoscope system comprising:
an optical fiber for guiding illumination light supplied from a light source section;
an actuator configured to displace an irradiation position of the illumination light emitted through the optical fiber; and
a controller configured to:
generate a driving signal including periodicity as a signal for driving the actuator,
supply the driving signal to the actuator via a predetermined signal line connected to the actuator,
detect presence or absence of occurrence of a trouble in the predetermined signal line by performing threshold determination based on either a current value of an electric current flowing in the predetermined signal line at predetermined timing in a period for one cycle of the driving signal or a voltage value of a voltage applied to the actuator at the predetermined timing, and
determine an occurrence of a trouble in the predetermined signal line based on a current value of an electric current flowing in a power supply line for supplying a power supply voltage to an operational amplifier connected to the actuator through the predetermined signal line and configured to amplify and supply the driving signal to the actuator.
